(19) United States
(12) Patent Application Publication
(10) Pub. No.: US 2007/0190048 A1
(43) Pub. Date: Aug. 16, 2007

(54) METHOD OF INDUCING PHYSIOLOGICAL PROCESS IN A BODY CAVITY AND A DEVICE FOR USING IT

(76) Inventors: Eric Lardinois, Haine-Saint-Paul (BE); Giorgio Maritan, Sant Pere de Ribas (ES); Wolfgang Petsch, Waterloo (BE)

Correspondence Address: WOLF, BLOCK, SHORR AND SOLIS-COHEN LLP, 250 PARK AVENUE, 10TH FLOOR, NEW YORK, NY, 10177, US

(21) Appl. No.: 11/566,844

(22) Filed: Dec. 5, 2006

(30) Foreign Application Priority Data
Dec. 6, 2005 (EP) 05447272.5

(51) Int. Cl. A61K 39/395

(52) U.S. Cl. 424/133.1

(57) ABSTRACT

This invention relates to a method of inducing an auto-immune reaction in a host in need of such inducement. The invention is characterised in that stressful stimuli are applied to the vaginal and cervical mucosa by warming, prior to sexual intercourse, said vaginal and cervical mucosa to a physiologically acceptable but stress inducing temperature degree.

The invention also relates to a device for using said method comprising a hollow body (1) provided with a heat source on the inside as well as with a means (13, 27) of removal of said hollow body from the vaginal or rectal cavity and a means (6, 8, 10) capable of exchanging heat originating from the hollow body.

### FIELD OF THE INVENTION

This invention relates, in general, to a method of inducing physiological processes in a body cavity and to a device for using it.

More precisely, the invention relates to a method of inducing physiological processes in the vaginal and/or rectal tractus more particularly for producing a contraceptive effect and/or a prophylactee effect through a stimulation of the immune system.

### BACKGROUND OF THE INVENTION

At the moment there are many female contraceptive methods including methods based on interpretation of the intravaginal temperature, hormone based methods or even mechanical methods including the placement of devices such as a diaphragm, an intrauterine device or a female condom.

All these methods and means are designed for use before coitus in order to prevent conception.

However, an emergency or post-coital contraception may be envisaged when no contraceptive means have been used under these conditions, or when the contraceptive means have proved to be deficient when an unwanted conception is probable. This type of contraception is designed to prevent fertilisation in order to prevent an unwanted pregnancy, and represents the latest stage at which contraception is possible before abortion.

At the moment, two types of post-coital contraceptive methods are known, one chemical making use solely of a progestative such as progestin, for example levonorgestrel or an oral estroprogestative combination for example including progesterone and a progestin such as levonorgestrel, and the other mechanical based on placement of an intrauterine device (IUD) such as a sterilet.

However, these methods do have undesirable side effects.

In this respect, it is emphasised that administration of hormones can cause appearance of different side effects such as nausea, vomiting, irregular vaginal bleeding, abdominal pain, vertigo, asthenia, mastodynia or headaches, while an intrauterine device should not be recommended for persons with high risks of sexually transmissible infections. It is known that insertion of such a device can lead to a pelvic infection capable of causing infertility if untreated, or even worse capable of forming a suitable site for invasion of the human immunodeficiency virus (HIV) if it is exposed. Such methods require help by the medical profession, particularly due to these disadvantages and undesirable side effects.

Consequently, the development of a non-toxic, non-hormonal, post-coital contraceptive method without side effects, making use of a single usable and/or reusable means and that can be put into place easily and preferably without any help from the medical profession, is of great interest.

It is known that the morphology of spermatozoids is vulnerable, unstable and sensitive to even a small excess temperature as well is to cellular and humoral immune system activation.

Thus, a temperature of 0.5° C. above the normal body temperature is already sufficient to disorganise their motility.

Furthermore, at a temperature of the order of 42° C. to 44° C., spermatozoids burst between the head and the tail preventing their motility, while at about 45° C. the head bursts irreversibly making them totally ineffective.

A male contraceptive method based on excess heat has been described in patent application DE 4 041 254 using a device in the form of a pouch heated to a temperature of 39° C. to 40° C., in other words slightly above the body temperature. According to this patent application, the pouch placed around the testicles disturbs production and motility of spermatozoids due to the increased temperature, thus producing temporary infertility in the man.

However, after coitus, the spermatozoids that had been subjected to such a treatment will be located in a completely different confinement medium from where they were in the testicles, because they will be affected by various factors and mechanisms inherent to the female genital sphere.

It is known, in fact, that spermatozoids deposited in the vaginal cavity may be guided to the fertilisation site by means of a thermal mechanism involving their movement towards the larger amount of heat emitted by the ovule fertilisation site.

In other words, after coitus, spermatozoids may start migration in the genital tract as a result of thermal signals and during this migration, acquire maturity necessary for fertilisation under the effect of various factors controlled by the female physiology.

For these reasons, the post-coital behaviour of spermatozoids output from testicles in which only the outside envelope was in contact with a device heated to a temperature as low a 39° C. to 40° C., and then brought into the presence of the vaginal sphere at a normal body temperature, can be no better than unpredictable. Also, the efficiency of such a contraceptive effect may be validly questioned. Consequently, the method described in the above mentioned patent application based on a slight increase in the temperature of the testicular confinement medium of the spermatozoids above the normal body temperature, does not appear to be a reliable contraceptive method.

At the moment, the Acquired ImmunoDeficiency Syndrome (AIDS) is a genuine challenge due to the threat that it represents, and the objective is to control and eliminate it by all possible means at each possible step in the transmission of the infection, particularly as soon as the virus is introduced into the host.

For example, it is known that a traditional prophylactic treatment can be considered after a high-risk sexual contact involving possible exposure to HIV. However, this must be undertaken immediately after the sexual contact or within 24 hours so as to benefit from its best potential. Nevertheless, there is no guarantee that viral infection will be avoided. After 72 hours, there is no longer any hope of the preventive treatment preventing progress of the virus.

However, an HIV inoculation immediately after sexual exposure to HIV may prove to be slow and therefore very weak, which could improve more efficiency of a single or multiple prophylactic treatment during this crucial period.

As explained below, viral replication is, however, capable of “memorising” mechanisms, that, throughout the evolution of the virus, lead to modifications to the virus in response to some external factors. Consequently, this memorisation phenomenon could provide an escape mechanism from anti-retroviral drugs and future vaccines.

It is known, in fact, that the introduction of HIV into a host initiates, as in the case of other viral infections, immediate response from the immune systeme as a whole, which in most cases partially controls the viral replication. Essentially innate immune response in HIV infection could provide the first level of defense that will prevent infection in some individuals and help control the infection in others.

Thus, the innate system of the host recognises HIV by the pattern of its viral components but, just when one shape has been recognised, HIV may balance viral fitness and change the shape of its antigen through random mutations. This allows the virus to chemically replicate and to eventually wear down the body's defenses by destroying the very cells necessary to coodinate an effective immune response.

A key feature of immune escape is therefore the ability of HIV to conceal itself from neutralizing antibodies that would otherwise bind the virus and block its entry into cells. In fact, HIV protects itself from antibodies by putting up a shield of constantly shifting sugars moieties. This shield may be contributting to the poor performance of candidate HIV vaccines.

### DETAILED DESCRIPTION OF THE INVENTION

Within the scope of the invention, a method has been found especially of contraception and prophylaxis, method based on the use of physiologically auto-induced heat shock proteins (HSP's) immunity to prevent pregnancy and to guard against common sexually transmitted diseases (STD's) including acquired immunodeficiency syndrome (AIDS).

Thus, it has been found that by leading a temperature increase, sufficient to increase the intravaginal and/or intrarectal temperature above the natural temperature, a cascade of biological barriers i.e. physiological or biological factors can be created that induce intravaginal and cervical and/or intrarectal “mucosal immunity”.

In fact, such a temperature increase is capable of inducing, through a release of HSP's, a trigger and/or stimulation of the immune system especially the innate immune system so creating a biological barrier contraceptive that shut down the cervix cavity and limits the migration or free movement of spermatozoids or a biological barrier against viruses, for instance that limits the transmission and replication of HIV.

Therefore, a first object of the present invention relates to a method of inducing, in the vaginal and/or rectal tract of a host in need of such inducement, physiological processes capable of disturbing factors which determine the evolution and/or development of normal biological processes affecting either pathogenic or non-pathogenic agents or affecting pathological or non-pathological physiological conditions of the host, said agents and physiological conditions of the host being present in said vaginal and/or rectal tract whereby heat is input into the whole vaginal tract including the vaginal cavity, its entrance and cervical mucosa and/or into the rectal tract to increase the intravaginal and/or into the rectal tract to increase the intravaginal and/or intrarectal temperature above the natural body temperature to a physiologically acceptable temperature inducing a non-injurious stress.

In accordance with another feature of the invention, the intravaginal and/or intrarectal temperature increases up to at least 55° C. usually up to 45° C.

Yet, in accordance with another feature of the invention, the pathogenic agents are sexually transmitted pathogens such as bacteria, bacterial-toxins, parasites and viruses for example HIV; the non-pathogenic agents are spermatozoids; the pathological physiological conditions are pre-cancers, non-invasive cancers and sexually transmitted diseases (STD's) and the non-pathological physiological conditions are an ovule fertilisation or a beginning of pregnancy.

So, the method according to the invention relies on an application of stressful stimuli provoked by a non-injurious stress inducing factor, i.e. heat, to vaginal and cervical mucosa and/or ano-rectal mucosa prior to or after sexual intercourse. As a consequence, cervical and vaginal mucosal cells and/or ano-rectal cells will induce and load on HSP's.

Yet, in accordance with a preferred aspect, the invention relates to a method of inducing an auto-immune reaction in a host in need of such inducement whereby stressful stimuli are applied to the vaginal and cervical mucosa by warming, prior to sexual intercourse, said mucosa to a physiologically acceptable but stress inducing temperature degree.

In accordance with another aspect of the invention, HSP's are induced inside vaginal and cervical mucosal cells in large quantities as a result of said applied stressful stimuli.

Yet, in accordance with a further aspect of the invention, few HSP's loaded mucosal cells are damaged as a consequence of the mechanical friction caused by sexual intercourse.

Furthermore, in accordance with another aspect of the invention, HSP's are released into extra-cellular space as a consequence of the damaged mucosal cells, said released HSP's, and their contact with other vaginal and cervical mucosal cells inducing an auto-immune response.

In accordance with a further aspect of the invention, trigger floods of anti-spermatozoid antibodies as well as antibodies against pathogens responsible for sexually transmitted diseases are released, in the presence of respectively spermatozoids and pathogens responsible for sexually transmitted diseases, as a result of the auto-immune response.

Furthermore, in accordance with another aspect of the invention, the anti-spermatozoid antibodies induce an immobilisation and/or destruction of the spermatozoids and the antibodies against pathogens related to sexually transmitted diseases induce a neutralisation of said pathogens.

In accordance with another aspect of the invention, the cervical mucosa is sensitised by antibodies and auto-immune response resulting in prevention of spermatozoids entry into uterine cavity.

HSP's, also called stress proteins, are a group of proteins that are present in small amounts in all cells in all life forms. They are also induced in very large quantities when a cell undergoes various types of environmental stresses like heat, cold and oxygen deprivation. Under normal conditions, HSP's act like “chaperones” of other protein families inside the cell. They make sure that the cell's proteins are in the right shape and in the right place at the right time. For example, HSP's help new or distorted proteins fold into shape, which is essential for their function, or shuttle proteins from one compartment to another inside the cell, or tansport old proteins to “garbage disposals” intracellular apparatuses. HSP's are also believed to play a role in the presentation of pieces of proteins (or peptides) on the cell surface to help the immune system recognize diseased cells.

When HSP's are found outside the cell, they indicate that a cell has become so sick that it has died and spilled out all of its contents. This kind of messy, unplanned death is called necrosis, and only occurs when something is very wrong with the cell. Extracellular HSP's are one of the most powerful ways of sending “a danger signal” to the immune system in order to generate a response than can help to get rid of an infection or disease.

In addition to an HSP's synthesis induced by heat, in accordance with the invention, any mechanical friction during sexual intercourse will cause some vaginal or cervical mucosal cells to rupture thus releasing large quantities of HSP's into vaginal cavity so that the presence of such extra-cellular HSP's, reinforced with their contact with other vaginal and cervical cells will produce an auto-immune reaction releasing an array of antibodies against both spermatozoids and other sexually transmitted pathogens and diseases. The induced auto-immune reaction will also sensitise the cervical mucosa thus preventing spermatozoids from entering into uterus.

The whole process of the invention so described results therefore in an instant active immunisation of vaginal and cervical mucosa against spermatozoids and common pathogens responsible for sexually transmitted diseases.

In accordance with a particular aspect of the invention, it has been found that a temperature increase sufficient to increase the intravaginal temperature above the natural temperature, essentially an increase in the temperature up to at least 42° C. to 45° C., and before fertilisation, causing direct and sufficient alteration to the morphology of spermatozoids to inhibit their migration to the fertilisation site and consequently acquisition of the maturity necessary for fertilisation during this migration, thus inducing a prevention of fertilisation, in other words a contraceptive effect.

This alteration of spermatozoids leading to their destruction may be produced and stimulated, as described above, by different factors in cascade in situ throughout genital sphere as a whole, in other words including the entrance to the vagina.

Furthermore, such an increase in the temperature inside the vagina as far as and including the cervix is found to be capable of satisfying a preliminary condition for early interruption of pregnancy or spontaneous abortion after coitus and/or after fertilisation, due to the creation of conditions unfavorable for maintaining the early fertilised ovule (zygote) at the transit stage that lasts about 14-16 days from the egg release site to the uterus implantation process site.

In addition, an increase of the intravaginal temperature of the same order before and after coitus was capable of creating conditions favourable to contraception following the occurrence of complex physiological processes capable of discouraging any step for movement of spermatozoids in the genital tract and more controlling implantation of the zygote in the decidua wall, thus causing its spontaneous expulsion from the uterus.

In particular, an extra input of heat in the genital tract before coitus already induces conditions particularly in the vaginal sphere, such that if it is necessary to increase the intravaginal temperature after coitus, the effective time available to provoke such a temperature increase after coitus would be shorter than if the temperature had not been increased before coitus.

Consequently, these various observations are the background for the proposal of a method of generating physiological processes capable of creating conditions unfavorable for initiation of an ovule fertilisation and/or a pregnancy in a woman necessitating these physiological processes, according to which heat is input into the entire vaginal cavity including its entrance and cervical mucosa, to increase the intravaginal temperature above the natural body temperature up to at least 42° C. to 45° C. so as to induce, in situ, said physiological processes capable of provoking disturbed biological processes which are:

a) after coitus and before fertilisation, disorganization of the motility and a direct alteration of the morphology of spermatozoids sufficient to inhibit their objective of migration to the fertilisation site and their possibility of acquiring the maturity necessary for fertilisation and thus produce a prevention of fertilisation i.e. a contraceptive effect,

b) after coitus and after fertilisation, a lack of keeping of the fertilised ovule in the uterus, optionally in the presence of one or several natural prostaglandins and/or one or several prostaglandin derivatives, so as to form a preliminary condition to early interruption of pregnancy, and even to produce an abortive effect,

c) before coitus, a prevention of fertilisation subsequent to a disturbance of the factors that determine the maturity of spermatozoids and a discouraged implantation of the zygote in the decidua wall, thus causing its expulsion from the uterus.

As already mentioned, an input of heat sufficient to cause an increase in the intravaginal and/or intrarectal temperature above the body temperature of 37° C., essentially an increase in temperature up to at least 55° C., is capable, of triggering in situ physiological processes or phenomena called “response to heat shock” before and after coitus characterised by various factors including synthesis of HSP's.

As also described above, these over expressed HSP's and/or heat stress proteins produced into vaginal and mucosal cells and/or rectal cells then play a greater protection role (“chaperones”) with regard to other polypeptide constituents of those cells, in that they are capable of stabilising proteins in an abnormal configuration and playing a role in folding and unfolding proteins in general. For example, there is the HSP70 heat shock protein that is of primordial importance, and the HSP27 protein that is highly expressed in women in the uterine endometrium, the vagina and the cervix

As a result of these over expressed HSP's, HIV is incapable of setting up adaptation mechanisms referred to above but on the contrary is affected by irreversible modifications that weaken it and make it vulnerable to the immune system. In other words HIV denounces its presence to the immune system as a result of a physiological processes induced, according to the method of the invention, by an appropriate warming unlike the method according to the above-cited German patent application DE 4041254 which appears to completely inadequate for a male contraception and/or to achieve prevention of STD's.

Approaches to induce firstly innate immunity appear obvious and form a first priority in health care. In other words, taking action before the infection occurs represents a non-negligible opportunity for preventing a pre-cancer, a non-invasive cancer or any viral proliferation throughout the genital sphere, and particularly in the vaginal cavity and the cervix.

Since HSP's are capable of acting on and controlling the immune system, in particular the innate system, an enhanced production of these proteins can provide valuable assistance to trigger and/or stimulate defense mechanisms of the organism and the immune system in general through, for instance, cytokines and interferon. In this respect, the HSP70 protein and other members of the HSP family is found to be capable of operating like an innate anti-HIV factor so as to limit its transmission and replication cycle.

In addition, the input of heat discussed above could trigger and/or activate the genital and/or ano-rectal immune system and particularly the innate genital and/or ano-rectal immune system, through shock and/or stress proteins, so as to generate a favourable response in the different female problems including irritations and also in process for prevention and even treatment of some cancers (pre-cancers and non-invasive cancers) such as cancer of the vagina, cervix, uterus or the ovaries, of even some infectious or viral diseases and particularly sexually transmitted diseases especially AIDS.

As a consequence, these different observations justify a proposal for a method capable of activating the immune system especially the genital and/or ano-rectal immune system so as to generate a favourable response in the cancer prevention and treatment process, essentially for pre-cancers and non-invasive cancers and infectious or viral diseases and particularly sexually transmitted diseases, especially AIDS in a woman requiring such a favourable response, method by which heat is input into the entire vaginal cavity including its entrance and cervical mucosa and/or into the rectal cavity to cause an increase in the body temperature up to at least 55° C. so as to induce triggering of HSP's and/or heat stress proteins in situ capable of stimulating said genital and/or rectal immune system.

Devices capable of increasing the temperature of a body cavity, and particularly the rectal or vaginal temperature, are already known.

Thus, patent GB 384 179 describes a device to increase or reduce the temperature of a body cavity including a metal support for a tubular element that passes through this support from one side to the other and for which the upper portion is designed to be inserted into the body cavity. This tubular element is closed at the end of its upper portion located on the side of the upper wall of the support and connected to a three-way valve through the other end located on the side of the lower wall of this support, this three-way valve itself being connected to an external heating or cooling source. Furthermore, a removable envelope may be placed above the upper portion of the tubular element and fixed to the upper part of the support through a screw ring and to a seal.

Similarly, patent U.S. Pat. No. 2,192,768 discloses a device designed to apply heat to the genital—urinary organs. This device comprises a cylindrical body divided into two separate chambers, one an entrance chamber and the other an exit chamber. A hollow double loop designed to be inserted into the body cavity connects the upper ends of these chambers together. Furthermore, this device includes two conduits, one connecting the lower end of the entrance chamber to hot water source to be inserted into the loops, the other connecting the lower end of the exit chamber to expel water out of the loops.

Furthermore, patent U.S. Pat. No. 3,170,465 describes a device for inputting a certain quantity of heat to different parts of the body and particularly the rectal or vaginal cavity. This device may be considered as being standalone because it is capable of producing this heat itself in situ from an exothermal reaction created by chemical compounds initially separated by a partition, then brought into contact, for example after breaking this partition.

No clarification is made about the nature of these chemical products or the type of reaction produced, which suggests that it is not reversible and consequently the device in question cannot be reused. Furthermore, it is not mentioned that this device is capable of inducing a contraceptive effect. Furthermore, a patient who would like to receive this device must remain immobile while it is being put into place, which must be done by qualified personnel.

Furthermore, patent application WO 2004/009004 discloses a device for therapeutic heat treatment of the vagina starting from a cylindrical shaped body with rounded soft edges, this cylindrical body comprising a removal cord fixed to one of its ends. This device, in fact the cylindrical body, contains a heat accumulating medium, the heat originating from an outside bath of warm water or being heated using a microwave oven.

However, although this device is reusable, it does not appear to be standalone because it is not capable of generating the required heat by itself in situ. Consequently, such a device requires an external heat input immediately before use, which can introduce some constraints.

In accordance with a further aspect, the invention provides a device for inducing an auto-immune reaction in a host for instance for putting into application the method of the invention described hereabove, device that can produce heat in the vaginal or rectal cavity, so as to increase the temperature and overcome the disadvantages or the deficiencies mentioned above, this device normally being useable and possibly reusable in complete safety, and that the user can put into place directly without any action by the medical profession being necessary.

To achieve this purpose, the device capable of raising the temperature of the rectal cavity and more particularly the vaginal cavity, is of the type comprising an oblong shaped hollow body for insertion into vaginal or rectal cavity, including a sidewall, an upper end cross wall and a lower end cross wall, this hollow body being provided with a heat source on the inside and a means of removal from the vaginal or rectal cavity on the outside, and is characterised in that:
the lower end cross wall of the hollow body is connected to the removal means by a means capable of exchanging heat between firstly a part of said hollow body and secondly either said removal means or at least a ring clamping said heat exchanger means or said removal means and at least a ring clamping said heat exchanging means, and the upper end cross wall of the hollow body optionally includes a means capable of exchanging heat between said hollow body and at least one ring clamping said heat exchanging means, or
the lower end cross wall of the hollow body is connected to the removal means and the upper end cross wall of the hollow body comprises a means capable of exchanging heat between said hollow body and at least one ring clamping said heat exchanging means,

such that the heat source generates a quantity of heat capable of increasing the temperature of the total vaginal or rectal cavity up to and including their entrance.

According to one particular and advantageous feature of the invention, the hollow body is connected to the removal means by a means capable of exchanging heat between said hollow body and said removal means, a ring possibly clamping said exchanging means.

According to another feature of the invention, the heat source generates a quantity of intravaginal or intrarectal heat sufficient to put the method described above into application, and particularly to prevent migrations into the vaginal tract, including the migration of spermatozoids, especially into the cervix and the upper fertilisation, and consequently, to produce a contraceptive effect.

According to another feature of the invention, the heat source generates a quantity of intravaginal heat capable of putting the method described above into application and particularly triggering and/or stimulating the female genital immune system. Thus, this quantity of additional heat is capable of different effects including triggering production of HSP's and/or heat stress proteins that play a role between innate immunity and the adaptation immune system through cytokines and interferon.

The hollow body of the device according to the invention with an oblong shape appropriate for intravaginal or rectal insertion, comprises an upper insertion end and a lower removal end at the opposite end provided with a heat exchanging means.

The shape, size and stability of this hollow body may be symmetric or asymmetric about the longitudinal central axis of the body.

In general, this shape will be approximately cylindrical, with a smooth and essentially soft outer surface with no sharp edges, advantageously rounded or with no sharp edges so as to avoid injury and trauma, for example such as the perforation of the vaginal canal or the rectal cavity during the insertion of the hollow body, an inflammatory syndrome caused by a foreign body or bacterial contamination.

However, the initial shape of this hollow body may be modified particularly with regard to its transverse axis so as to intimately match the shape of the vaginal or rectal cavity and particularly the upper vaginal cavity located at the cervix. For example, the cylindrical longitudinal wall of the hollow body may be provided with one or several increases in size or reductions in size, over all or some of its height. These modifications to the shape are arranged either regularly or irregularly around the entire periphery of the hollow body. For example, the region of the periphery of the hollow body located approximately at mid-distance between the insertion and the removal ends may include a reduction in size such that the perimeter is equivalent to 95% to 75% of the perimeter of the insertion end and/or the removal end.

Furthermore, the insertion end and/or the removal end cross walls may comprise at least one increase in size and at least one reduction in size, independently of each other. These modifications may relate to all or some of the surface of these end walls.

The over all length and width of this hollow body are advantageously determined appropriately taking account of the different expansion coefficients of the materials from which the envelope that delimits it is made. Thus, the length of the hollow body in question, in other words the overall length separating the ends of the two cross walls is usually less than or equal to 150 mm, preferably 65 mm, and the overall width of this same hollow body is usually less than or equal to 50 mm, preferably 23 mm.

The hollow body of the device according to the invention comprises an outside envelope formed from a film of appropriate materials with different coefficients of thermal expansion. These combined films may be associated by being fixed together and/or sealed so as to originate in a desirable expansion profile of the assembly such that for each increase in temperature inside the hollow body, the expansion of the outermost film(s) further inside this hollow body.

Normally, this outside envelope is about 0.01 to 0.8 mm thick and is preferably about 0.02 mm to about 0.5 mm thick, and is made from one or several stable and strong but supple, flexible and elastic materials, preferably capable of “self-healing”. In this respect, the material described in patent application WO 2005/046540 and marketed under the FFLEXX® trademark (Wellcare Products Group) is an ideal material, that will enable a particularly efficient seal.

Materials that can be used to make the envelope of the hollow body will be non-toxic and biocompatible, and will also be capable of resisting any variation of temperature and the different components used in the supersaturated solution in complete safety, particularly to prevent their diffusion through this envelope. In order to achieve the required safety, these materials should also be capable of resisting the different daily physical stresses caused by internal and external pressure, particularly during manual removal of the device represented essentially by the hollow body. In this context, the safety level should be about 200 kg.

Thus, the envelope of this hollow body may be formed from a film of polymeric material or another conventional flexible material capable of containing heated liquids, for example rubber or silicone.

Polymeric materials that can be used can be selected from among polyethylene, polypropylene, polyamide, polyester, polyvinyl chloride, polyvinylidene chloride, polystyrene, polyurethane, or a saponified or non-saponified copolymer of ethylene-vinyl acetate. Furthermore, these materials can be associated with woven or non-woven fabrics suitable to come into contact with heated liquids.

Depending on needs, the envelope of the hollow body is made from a material with a spongy or porous structure that can be coated and/or impregnated with biocompatible agents or may contain such agents chosen particularly from among spermiostatic, spermicide, anti-infectious, virucide, bactericide agents, natural prostaglandins, and derivatives of prostaglandins, or in general chosen from any medication or other agent, particularly that can be used in the rectal or vaginal pathway or any composition containing such agents.

Furthermore, according to one particular and advantageous feature, the hollow body is surrounded by a sheath or a removable envelope made of a material that can be coated and/or impregnated with one or several biocompatible agents, medicated or non-medicated, chosen from among the agents and compositions mentioned above.

More particularly, the hollow body in question may be used as a support for such an envelope, itself useful as a medication support that can be administered by the rectal and/or vaginal pathway. In particular, this relates to medication for which resorption by the rectal or vaginal mucosa is improved when the temperature at this absorption site is increased.

Depending on needs, the envelope in question fitted removably on the hollow body and administering a medicated or non-medicated agent chosen from among the family mentioned above, may easily be removed and replaced by a similar envelope carrying another agent belonging to this family.

The heat exchanging means located at the lower cross wall may usually include a lower prominence extending from it above which there is a protuberance extending from or added onto this prominence, and in this protuberance the removal means may slide freely or may be partially integrated.

When the removal means is capable of sliding freely, this protuberance may be in a single part but advantageously it may be in several parts with the essential purpose of increasing the outside surface area of the exchanger and consequently heat exchanges.

As an example, this heat exchanger may comprise a protuberance, one portion of its wall being common with a portion or all of the lower prominence of this hollow body.

Consequently, according to one particular embodiment, the protuberance inside which the removal means slides may be covered by a removable cap, usually made of a material that is a good conductor of heat, that may be in contact only with this protuberance or also with the lower prominence of the hollow body.

Furthermore, when the removal means is partially integrated into the protuberance forming part of the heat exchanging means, a maximum portion of the surface of this removal means is in contact with this protuberance itself in contact with a maximum portion of the lower prominence.

According to one particular embodiment, the hollow body, in addition to the heat exchanging means at its lower cross wall, comprises an additional heat exchanging means located at its upper cross wall, this additional means comprising an upper prominence output from this wall or added onto it.

However, according to another embodiment of the invention, the hollow body does not have a heat exchanger at its lower cross wall.

The capsule removal means is usually composed of a manual means such as a removal cord, normally made of a supple material so as not to interfere with normal activities of daily life. In general, it is composed of a single strand that may or may not be hollow, terminating by a distal loop to facilitate removal from the hollow body or preferably a hollow or non hollow strand in the form of a ring, one or the other of these strands being attached directly or indirectly to the hollow body.

According to another embodiment, the removal means consists of a removal tab, possibly being provided with a hollow or solid peripheral rim.

This removal means also forms an additional security element to automatically protect the zone located between external female genital organs and the vaginal cavity. Furthermore, this removal means is visible from the outside at the genital organs, so that the user can periodically check that it is present or in the correct position.

According to another feature of the invention, this removal means may be impregnated and/or coated with one or several biocompatible agents, medicated or non-medicated, chosen from among spermiostatic, spermicide, anti-infectious agents, virucides and bactericides for example formulated in the form of a cream.

When it is hollow, the removal means advantageously encloses one or several heat transfer agents such that the release rate of the biocompatible agents in question can be controlled when this removal means is efficiently heated. These agents, that may be in solid, liquid or gas form, are selected from among materials capable of good conduction and/or convection of heat collected from the heat exchanger. When it is in solid form, the heat transfer agent may comprise one or several supple metallic wires that are good conductors of heat. These wires advantageously occupy the entire hollow part inside the removal means and as a result may be considered as being embedded in the mass of this removal means. When this heat transfer agent is a liquid, it will be chosen so that its boiling temperature is greater than the boiling temperature of the liquid into which the device according to the invention will be heated after use.

Such liquid agents, some of which have been described for example in patent U.S. Pat. No. 5,417,276, will be thermally stable, safe and non toxic and will have sufficient viscosity to prevent diffusion through the removal means. Furthermore, when this heat transfer agent is in liquid form, it will preferably be non-volatile for safety reasons.

Furthermore, according to another feature of the invention, the heat exchanging means in the lower cross wall and the heat exchanging means in the upper cross wall when there is one, is (are) preferably clamped by a ring adjacent to this wall, and particularly a circular ring.

However, according to one particular embodiment, only the upper cross wall comprises an adjacent ring clamping the heat exchanger, the heat exchanger at the lower cross wall not being provided with such a ring.

Advantageously, this ring is configured so that it does not prevent flow of vaginal fluids that participate particularly in heat transfer and transfer of immunity factors originating from vaginal and cervical secretions.

On the contrary, such a ring tends more to encourage drainage of these fluids without any blockage of them along the vaginal mucosa. These fluids, when they carry one or several medications, enable better distribution and better absorption of medication by the vaginal or cervical mucosa. Furthermore, these fluids assure tissue integrity so as to maintain a healthy vaginal environment despite cyclic variations. This is why a toroidal shaped ring is preferred in particular.

After placement of the device according to the invention, this ring will be located close to the vaginal orifice when it is adjacent to the insertion end if this ring is provided will be located in the immediate vicinity of the cervix.

This ring, in relation with the heat exchanging means, may be coated and/or impregnated with biocompatible agents, medicated or non-medicated, or it may contain other such agents chosen among spermiostatic, spermicide, anti-infectious agents, virucides, bactericides, natural prostaglandins and prostaglandin derivatives. When it is impregnated with these biocompatible agents or when it contains them, the ring will allow these compounds to diffuse due to its spongy or porous structure provided for this purpose. However, these prostaglandins and/or prostaglandin derivatives, due to their abortive effect, will be located specifically near the insertion end of the hollow body.

Alternately, the removal and insertion ends may each be provided with a toroidal shaped ring as above, coated and/or impregnated with biocompatible agents as described above.

According to an additional feature of the invention, the heat source corresponds to an in situ heat generating system capable of increasing the temperature of a body cavity up to at least 55° C. Thus, according to one particularly advantageous embodiment, this in situ heat generating system comprises a supersaturated and supercooled saline solution and a means of triggering exothermal crystallisation of the salt(s) of the solution. In particular, this heat generating system comprises a supersaturated and supercooled saline solution formed from a solution of at least one inorganic compound in an aqueous medium containing an alcohol, and a mechanism to trigger the required crystallisation and the concomitant production of heat.

The inorganic compound is usually chosen from metal acetate, preferably alkaline metal acetate or an alkaline earth acetate such as sodium acetate and a metal nitrate and preferably an alkaline metal or alkaline earth nitrate such as calcium nitrate, whereas the alcohol is usually a polyalcohol chosen from among ethylene glycol and glycerol.

Furthermore, the solution in question may particularly advantageously contain as agent particularly capable of prolonging the validity of the device according to the invention such as an amine, for example aniline or any other agent capable of maintaining equilibrium of the recrystallisable super saturated solution and particularly nano particles.

This recrystallisable solution may be obtained using conventional methods including preparation of an aqueous solution of the inorganic compound, for example in distilled water, addition of an alcohol to this solution while stirring the mix and then addition of an aniline also while stirring the resulting medium.

The triggering means includes a triggering system or a trigger and possibly one or several support means and/or protection means. The triggering means, composed of at least one trigger diaphragm, is capable of having two extreme configurations in which it can be bent by an instantaneous displacement, causing an oscillation wave inside the closed internal heat production system and the concomitant start up of progressive exothermal crystallisation.

Due to an oscillation function with a single basic frequency, this trigger diaphragm is also capable of automatically resuming its initial configuration after any action that caused its deformation, if necessary so as to restart a new cycle following another applied deformation.

This “memory effect” exists in some metallic materials, and can be provoked when the materials are subjected to a heat shock, particularly a heat shock generated by the exothermal crystallisation reaction of the salt(s) and/or by the redissolution process of the salts in question during external input of heat, particularly using boiling water.

Different types of sudden deformation trigger diaphragms that can be used in the device according to the invention, are known and have been described in the state of the art. For example, patent EP 0 464 092 that discloses a trigger diaphragm, in the form of a flexible narrow ribbon or strip, advantageously provided with grooves or perforated with thin slits at intervals, to maximise the wave oscillation reaction. This diaphragm, for which the peripheral edge is advantageously circular or elliptical in shape, has a convex central portion normally the same shape as the outside edge of the diaphragm in question.

Furthermore, this diaphragm for which the outside surface is convex, in other words the surface on which a pressure force must be applied, may comprise one or several usually V-shaped grooves, for which the point is facing towards the outside surface. Deformation of the diaphragm under the action of pressure causes a wider opening of the grooves and then when it returns to its initial position, narrowing of this opening and a resulting compression of the saline solution, consequently initiating its crystallisation.

However within the scope of this invention, the proposal includes a triggering means comprising different characteristics that, when taken as a whole, are particularly advantageous. This triggering means, adapted to the restricted dimensions of the device according to the invention, combines high performance, resistance, long life and ease of use.

Thus, according to a first advantageous characteristic of this trigger means, the thickness of the diaphragm varies from its centre towards its outside periphery as a function of the different tension zones thus created during when axial pressure is applied on this diaphragm to cause its deformation. These different thicknesses will have the advantage of uniformly distributing internal tensions thus generated in this diaphragm. Consequently, overtensioned zones can be minimised thus contributing to greater reliablility in the mechanical strength of the diaphragm in question. Furthermore, the thickness of this diaphragm may also vary depending on the viscosity of the recrystallisation medium in which it resides.

Consequently, this diaphragm configuration thus described enabling greater resistance to tensions applied to it, will have an excellent influence on its efficiency and the duration of its validity.

Thus, a device according to the invention comprising such a diaphragm or triggering device combined with a supersaturated saline solution containing an amine may be used reliably hundreds of times for months or years.

According to another particular characteristic of the invention, the curved portion of the trigger diaphragm comprises three slits of equal length that extend symmetrically from its centre, consequently in directions separated from each other by 120°, each terminating in an orifice, and particularly a circular or elliptical orifice. These slits that may be made by laser are usually between 0.1 and 0.5 mm wide, for example 0.3 mm. However, the trigger diaphragm that initiates the oscillation wave forms a physical system that can vibrate independently of the slits in question when it is subjected to an outside force.

The orifices, that are preferably similar, are wider that the slits and in particular their function is to absorb tensions developed when applying pressure on this diaphragm to deform it. In this embodiment, the edges of the slits are rounded or bevelled to prevent any operational friction and even any contact between them when in stable equilibrium (in other words when the diaphragm is at rest), and when in a deformation position (in other words during a deformation), and when in unstable equilibrium (namely at the end of the deformation travel).

According to another characteristic of the invention, the slits are configured so that during deformation of the diaphragm under the effect of pressure, they change from a closest together position causing compression of the saline solution between said slots with sufficient release of heat to initialise exothermal crystallisation of the salt(s) in the solution.

Furthermore, according to one particularly advantageous characteristic of the invention, the trigger diaphragm comprises a curved portion in the general shape of an equilateral triangle with sides curved slightly inwards and with rounded vertices. For reasons of convenience, this triangle will be called a “curved triangle” in the following.

In particular, such a curved triangle has the advantage that it enables an excellent distribution of the different forces developed when pressure is applied on this curved portion from its convex face.

Furthermore, according to another advantageous characteristic of the invention, the curved portion of the trigger diaphragm comprises three similar grooves located between the orifices and on a circumference with the same centre as this curved portion.

Similarly according to an additional characteristic of the invention, the trigger diaphragm comprises a non-peripheral rim outside the curved portion, extending transversely both to the concave side and the convex side of this diaphragm.

In particular, this rim has the advantage that it reinforces the equilibrium and strength of the diaphragm and absorbs energy and tensions generated during pressure on the diaphragm.

According to another characteristic of the invention, the trigger diaphragm is made from a shape memory alloy, essentially from stainless steel, an Ni/Ti alloy or a combination of the two, for which the heterogeneous structure is composed of particularly fine crystals.

Thus, this trigger system is usually made from a flexible non-corrosive material, preferably stainless steel, possibly combined with a Ni/Ti alloy and with eutectic characteristics. This steel is advantageously treated during fusion so as to create a heterogeneous mass during fusion formed from extremely fine crystals, and then cooled particularly slowly so as to maintain this heterogeneous structure composed of extremely fine crystals.

Consequently, the trigger diaphragm is made from a material with a structure identical to its structure during fusion, which assures very long life and reliable security.

According to another particularly advantageous characteristic of the invention, the trigger means comprises a trigger diaphragm, at least three support stands and two guide and/or protection elements, both being fixed to the trigger diaphragm.

These support and guide elements, with practically the same shape and dimensions as the diaphragm, are fixed to the diaphragm through at least three support stands and are located on each side of this diaphragm, the support and/or protection element on the concave side, the guide and/or the protection element on the convex side.

Considering the restricted dimensions of the trigger system, the support element and the guide element contribute particularly to determining the position of this system in the hollow body, for example when this position has to be determined by feel only.

According to different embodiments, the trigger system may be single or multiple, in other words it may comprise a single strip or on the contrary it may comprise several strips, for example two axially aligned strips.

Regardless of what configuration is adopted for the trigger system according to the invention and in particular due to its restricted dimensions, its actuation will require the use of only two fingers of one hand of a user.

The trigger, that is inserted into the hollow body, can float freely in the recrystallisable solution. This is why this trigger, and particularly the trigger diaphragm, is free of sharp edges to prevent perforations or tearing of the envelope of the hollow body. However, this diaphragm may be provided with annular rim around its periphery so as to increase the necessary safety. Furthermore, the trigger may be positioned in a fixed manner at any location on the inside of this hollow body. Advantageously, this trigger may be inserted in the end opposite to the end of insertion into the vaginal canal, in other words close to the removal end, so as to protect it from any unpredictable triggering of the exothermal saline crystallisation due to an uncontrolled external force. In particular, the trigger is located along the longitudinal axis of symmetry of this hollow body.

The invention will be better understood and other purposes, characteristics and advantages of it will become clearer after reading the explanatory description given below with reference to the appended figures given only as example illustrating different embodiments of the invention.

### BRIEF DESCRIPTION OF THE DRAWINGS

FIG. 1 is an exploded view of a device according to the invention, to increase the temperature of the vaginal cavity,

FIG. 2 is a plan view of a diaphragm for triggering a crystallisation,

FIG. 3 is a perspective view of the diaphragm in FIG. 2, showing the lines of force,

FIG. 4 is a vertical sectional view of the diaphragm in FIG. 2,

FIG. 5 is a perspective view of a means of triggering a crystallisation,

FIG. 6 is a sectional view of another embodiment of the device according to the invention,

FIG. 7 is a perspective view of the device in FIG. 6,

FIG. 8 is a horizontal sectional view of another embodiment of the device in FIG. 6,

FIG. 9 is a vertical sectional plan view of an embodiment of the device in FIG. 8.

### DESCRIPTION OF SPECIFIC EMBODIMENTS

### Example 1

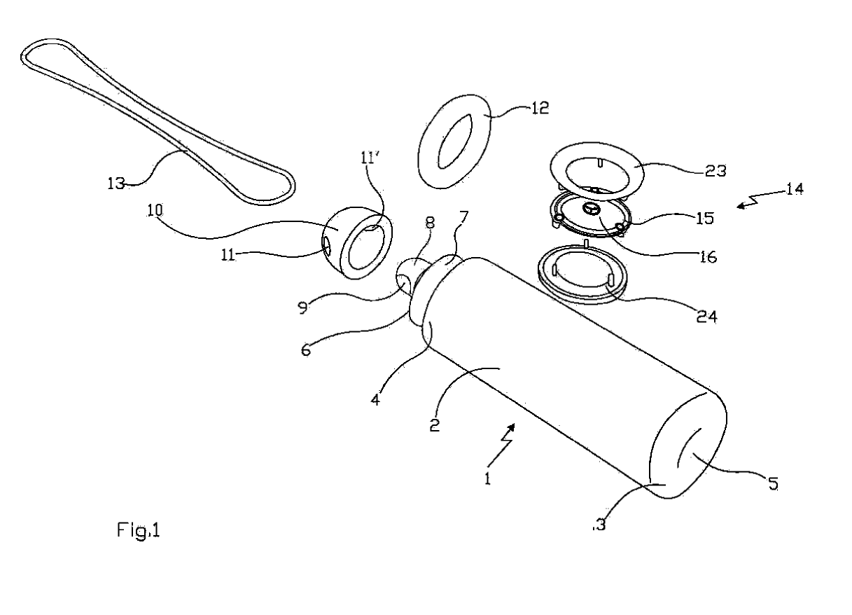

As shown in FIG. 1, the device according to the invention comprises an elongated hollow body 1 with a length of about 65 mm. This hollow cylindrical body with a diameter of about 19 mm is formed from an approximately 0.1 mm thick wall 2, with strongly rounded circular edges.

This hollow body is provided with two ends, each closed off by a cross wall, one wall 3 being the end inserted into the vaginal canal, the other 4 being the end at which it is removed. The end 3 is also provided with a reduced diameter 5, also approximately circular.

Furthermore, the wall 2 extends in a circular and preferably hollow prominence 6 provided with a radial groove 7, this prominence terminating in a protuberance 8 in the form of an attachment eyelet through which an orifice 9 passes. The assembly consisting of the wall 2, the grooved prominence 6 and the eyelet 8 is made from a stable non-toxic material, usually in a single piece. Advantageously this assembly may be made by moulding, for example using polyethylene, and covered with a fine film of a non-toxic, thermally stable heat conducting material, particularly a metallic material.

A cap 10 is provided above this eyelet comprising two orifices 11 and 11′ which, when this cap is placed on the eyelet 8, are located facing the orifice 9. This cap, which is also covered with a heat conducting material, completes the heat exchanging system already composed of the prominence 6 and the eyelet 8.

The assembly thus formed also comprises a toroidal ring 12 on the outside, preferably made of a spongy structure surrounding the prominence 6 and closely matching the shape of the groove 7, and secondly a removal cord 13 also made of flexible polyethylene in the form of a ring passing through the orifices 11, 9 and 11′ capable of sliding freely in the orifices. The toroidal ring 12 and the cord 13 are advantageously covered by a spermiostatic compound and/or spermicide if necessary, and also if necessary a medication such an anti-infectious and/or antiviral agent and/or one or several prostaglandins or prostaglandin derivatives.

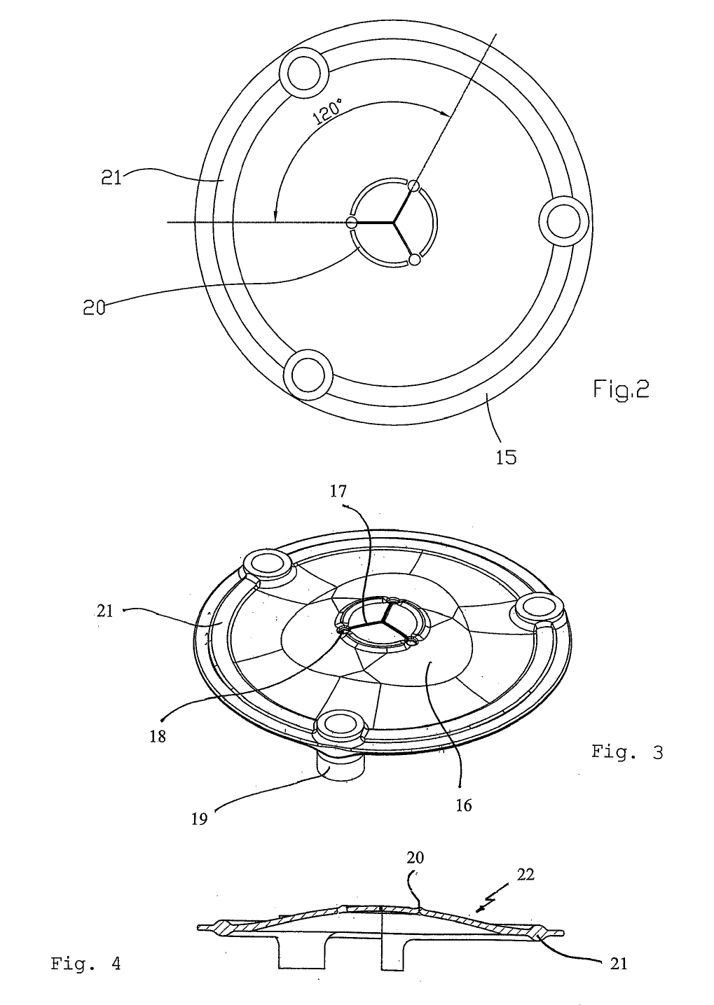

FIG. 1 also shows the different parts of a trigger 14. As shown in FIGS. 2 and 3, this trigger is essentially composed of a diaphragm 15 or a thin circular strip made of stainless steel comprising a Ni/Ti alloy, from which a curved portion 16 emerges in the general form of a curved equilateral triangle. This triangle is centred on this lamella and is materialised in the form of a solid line in FIG. 3.

This FIG. 3 also shows three identical slits 17 separated from each other by 120° and each terminating in a circular orifice 18, each leading outwards from the centre of the lamella 15, and each terminating in a circular orifice 18. Advantageously, the edges of these slits are parallel and bevelled and the orifices are identical.

Furthermore, this diaphragm is provided with three support stands 19. These support stands are partially recessed starting from their ends and are globally cylindrical in shape and extend on each side of the diaphragm concerned. It will also be noted that the centres of these support stands, each located on a circumference concentric with the diaphragm and at the intersection with a median of a side of the triangle in question, are separated from each other by 120°.

FIG. 2 also shows three grooves 20 in the form of three identical portions of the same concentric circumference of the diaphragm. These grooves in the arc of a circle are arranged symmetrically with each other and define three identical arcs of a circle forming separation zones, the centre of each zone being positioned on a radius passing through the vertex of the curved triangle. Thus, these centres are also separated from each other by 120°.

It can be also observed in FIG. 4 that the grooves open on the concave side of the diaphragm, and in FIGS. 2 and 3 that the centres of the circular stands are located at an annular rim 21 reinforcing the triggering system.

This rim, concentric with the diaphragm, extends symmetrically on each side of it. Thus, a force resulting from any pressure applied at the centre of the convex surface of such a fully symmetrical configuration diaphragm is distributed into six components with equal intensity along six axes separated from each other by 60°, the resulting force at the rim being zero.

Furthermore, it can be observed that in FIG. 4, the thickness of the diaphragm varies from the groove 20 as far as the rim 21, and that it is thinnest at 22.

The trigger diaphragm thus described is completed by two washers 23 and 24 that can be seen in FIGS. 1 and 5, each acting as a support and/or a guide and/or a protection shield. These washers are composed of a polyethylene circular wall tapering outwards, making them bell-shaped. Each of them is provided with three studs (not visible) on its globally concave lower face, that can engage in the recesses of the support stands of the diaphragm such that the diaphragm is held in the immediate proximity of the washers 23 and 24.

After reading the above, it can be easily understood that after coitus, when a contraceptive effect is still required before fertilisation, all that is necessary is to insert the hollow body into the vaginal cavity through its insertion end 3 and then to push it manually or using an applicator, to the neighbourhood of the cervix or inside the cervix. Firstly, crystallisation of salts dissolved in the enclosed medium in this hollow body will have been initiated so as to provoke production of a heat release. This is done by applying sudden pressure on the trigger system 15 at the centre of the portion 16 followed by relaxation of the trigger system, using two fingers with one on the convex surface of the diaphragm and the other under the washer 23.

The curved diaphragm will deform suddenly under the effect of this pressure. During this deformation, the edges of the slits 17 initially at a distance from each other, can get closer to each other to move through a maximum proximity position and then move away from each other to the end of the deformation movement distance. When this manual pressure is released, this diaphragm immediately and automatically returns to its primitive shape. However when they are at the minimum distance from each other, extra pressure is generated between the edges of the slits 17 with the release of sufficient heat to provoke initiating crystallisation of the compressed saline solution. This crystallisation continues then causing a release of heat sufficient to reach 38° C. to 55° C. inside the vaginal cavity. The manual search for the trigger system immersed in the saline solution in the hollow body is also facilitated by washers 23 and 24 for which the bell-shaped configuration makes it possible to guide two of the user's fingers as far as the centre of this diaphragm.

Furthermore, correct insertion of the hollow body into the vaginal cavity can be easily controlled when the toroidal ring 12 is located at the protuberance 6, in that this ring should be located in the vagina at its entrance, the cord 13 being also partially inside the vagina.

When the hollow body is removed as soon as the exothermal reaction is terminated, or for any other reason, all that is necessary to release the hollow body from the vaginal cavity is to apply a tension force on the cord 13.

If necessary, the device according to the invention may be reused after having brought the crystallised solution back into the liquid state by heating this crystallised solution, for example in hot water.

### Example 2

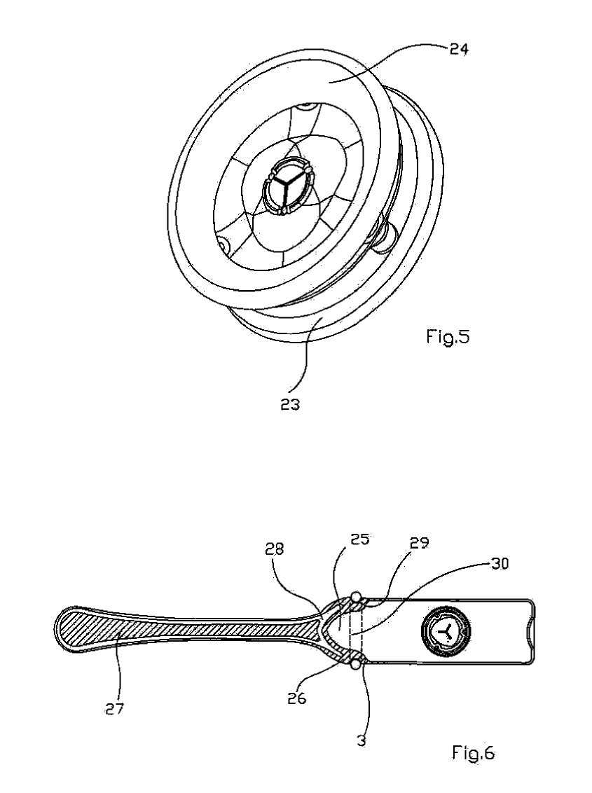

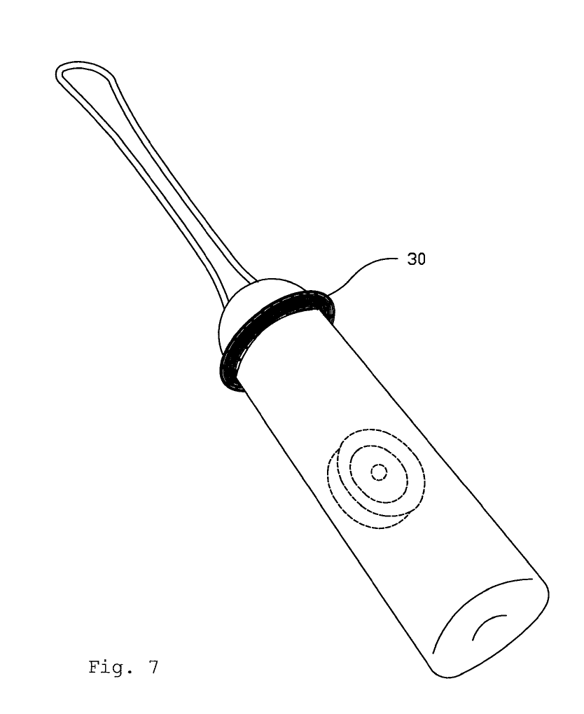

Another embodiment of the device described with reference to FIG. 1 is shown in FIGS. 6 and 7.

In this variant, the assembly formed by the removal means and the heat exchange means is configured differently.

Thus, FIG. 6 shows a circular prominence 25 corresponding to an extension of the lower end cross wall 3, this prominence being provided with a protuberance 26 above it that is prolonged by a removal tab 27.

A chamber 28 that is globally tubular in shape is arranged in this protuberance and this tab. As can be seen, this chamber runs along the inner periphery of the tab 27 and approximately matches the inner contour of the protuberance 26.

Furthermore, a radial groove 29 in the protuberance contains a torodial ring 30. This hollow ring is composed of an envelope that can be seen in FIG. 7, provided with a multitude of small orifices or pores capable of enabling slow diffusion towards the outside of a container located inside the ring.

### Example 3

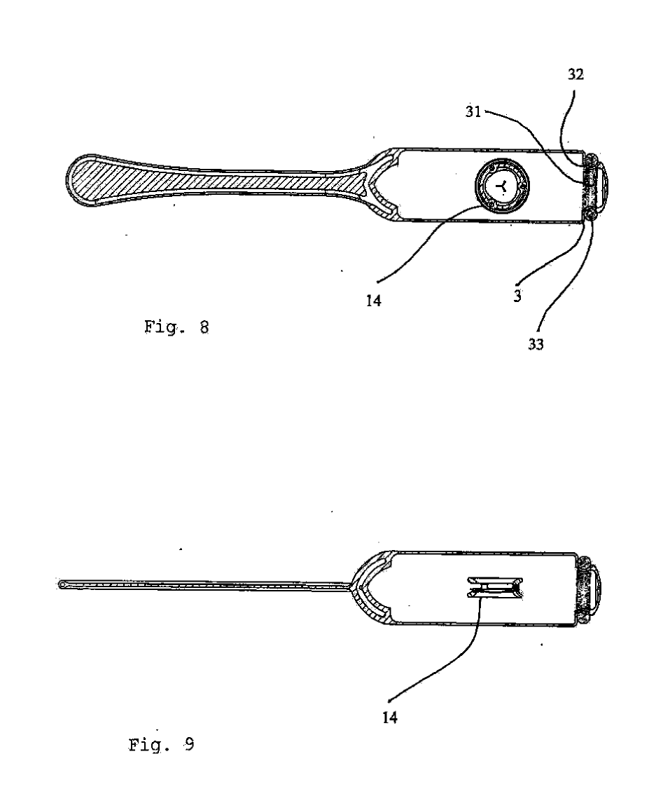

Another embodiment of the device described with reference to FIGS. 6 and 7 is show in FIGS. 8 and 9.

The assembly consisting of the toroidal ring is positioned differently in this variety.

Thus, FIG. 8 shows a circular prominence 31 corresponding to an extension of the upper end cross wall 3. Furthermore, a radial groove 32 in this protuberance contains a toroidal ring 33 similar to the ring 30, in other words hollow and composed of an envelope provided with pores useful for slow diffusion towards the outside of a container located inside the ring, for example a composition containing prostaglandins or prostaglandin derivatives.

FIGS. 8 and 9 also show the trigger 14 arranged inside the hollow body filled with a recrystallisable saline solution, in other words an aqueous solution of sodium acetate in ethylene glycol containing aniline.

The device according to the invention was found to be capable of generating a sufficient quantity of heat in situ, in other words in the vaginal cavity and as far as the cervix, or in the rectal cavity in order to increase the intravaginal or intrarectal temperature up to at least 55° C., at to a physiologically compatible rate during a sufficient time period to obtain the desired resulting effects.

Furthermore, sufficient addition of heat in particular to initiate a contraceptive effect can be obtained in less than 5 minutes after insertion of the device according to the invention into the vagina, when the saline solution has began to crystallise exothermally.

Due to its ability to cause intravaginal or rectal hyperthermia, the device according to the invention can also be used to produce other beneficial physiological effects, and particularly the production of HSP's or heat stress proteins and acquisition of a state called “heat tolerance” state, proteins for which the advantage is described in detail above. Such events generally begin to occur at about 43° C.

For example, a moderate local hyperthermia (about 42° C. or 43° C.) was recorded for 15 minutes, and in particular provokes an increase in HSP's for which cellular synthesis continues for 1 to 2 hours after this physiological heat shock.

Furthermore, this device according to the invention has an undeniable advantage due to the presence of a heat exchanger that participates in assuring that the temperature increase is uniform throughout the entire vaginal or rectal cavity. Unlike previous devices, the immediately at the entrance to the vagina or rectum and even at the external genital organs due to the removal means when it contains a heat transporting fluid.

It is of overriding importance when the objective is to destroy spermatozoids so as to initiate a contraceptive effect or to counteract propagation of a virus such as HIV, to be able to assure that no traces of these spermatozoids remain even inside the vagina when the device according to the invention is extracted from the vagina.

Furthermore, the device according to the invention has shown that it provides an excellent support for transporting different types of medicated or non-medicated biocompatible agents, through the envelope or cross walls of its hollow body. To achieve this, the device according to the invention has an undeniable advantage in that it can be used to place a sheath on the hollow body supporting medicated or non-medicated biocompatible agents, this sheath being removable and interchangeable with similar support sheaths.

1. A method of inducing an auto-immune reaction in a host in need of such inducement, comprising the step of: warming vaginal and cervical mucosa, prior to sexual intercourse, to a physiologically acceptable but stress inducing temperature degree, whereby stressful stimuli are applied to the vaginal and cervical mucosa.

2. A method according to claim 1, whereby warming induces the production of heat shock proteins inside vaginal and cervical mucosal cells in large quantities.

3. A method according to claim 1, whereby few mucosal cells loaded with heat shock proteins are damaged as a consequence of the mechanical friction caused by sexual intercourse.

4. A method according to claim 1, whereby heat shock proteins are release into extra-cellular space is a consequence of the damaged mucosal cells, said released heat shock proteins and their contact with other vaginal and cervical mucosal cells inducing an auto-immune response.

5. A method according to claim 1, whereby, as a result of the auto-immune response, bugger floods of anti-spermatozoid antibodies and antibodies against pathogens responsible for sexually transmitted diseases are released in the presence of respectively spermatozoids and pathogens responsible for sexually transmitted diseases, so that spermatozoids are immobilised and/or destroyed and pathogens are neutralised.

6. A method according to claim 1, whereby cervical mucosa is sensitise by anti-spermatozoid antibodies and the auto-immune response, which results in prevention of spermatozoids entry into uterine cavity.

7. A method according to claim 1, whereby an instant active immunisation of vaginal and cervical mucosa against spermatozoids and pathogens responsible for sexually transmitted diseases including human immunodeficiency virus is provided.

8. A usable and/or reusable device for inducing an auto-immune reaction in a host, in a host, of the type comprising an oblong shaped hollow body (1) for insertion into the vaginal or rectal cavity, including a sidewall (2), an upper end cross wall (3) and a lower end cross wall (4), this hollow body being provided with a heat source on the inside and a means (13, 27) of removal of said hollow body from the vaginal or rectal cavity, whereby:
the lower end cross wall of the hollow body is connected to the removal means by a means (6, 8, 10) capable of exchanging heat between firstly a part of said hollow body and secondly either said removal means or at least a ring (12, 30) clamping said heat exchanger means, or said removal means and at least a ring clamping said heat exchanging means, and the upper end cross wall of the hollow body optionally includes a means (31) capable of exchanging heat between said hollow body and at least one ring (33) clamping said heat exchanging means,
or
the lower end cross wall of the hollow body is connected to the removal means and the upper end cross wall of the hollow body comprises a means (31) capable of exchanging heat between said hollow body and at least one ring (33) clamping said heat exchanging means,
such that the heat source generates a quantity of heat capable of increasing the temperature of the total vaginal or rectal cavity up to and including its entrance.

9. A device according to claim 8, whereby the hollow body is connected to the removal means through a means capable of exchanging heat between said hollow body and said removal means, a ring optionally clamping the heat exchanging means at the lower end cross wall.

10. A device according to claim 8, whereby the hollow body:
either comprises an envelope made of a material with a spongy or porous structure, that can be coated and/or impregnated with biocompatible agents, medicated or non-medicated
or is surrounded by a removable sheath made of a material that can be coated and/or impregnated with biocompatible agents, medicated or non-medicated.

11. A device according to claim 8, whereby the removal means corresponds to a removal cord (13) formed from a hollow or solid strand in the form of a ring or a removal tab (27) optionally including a hollow or solid peripheral rim, this removal cord or removal tab being optionally coated and/or impregnated with one or several biocompatible agents, medicated or non-medicated, chosen from among spermiostatic, spermicide, anti-infectious agents, virucides and bactericides.

12. A device according to claim 8, whereby the means capable of exchanging heat comprises a lower prominence (6, 25) originating from the lower cross wall terminating in a protuberance (8) originating from this prominence or added onto it, protuberance in which the removal means can slide freely or be partially integrated.

13. A device according to claim 8, whereby the means capable of exchanging heat comprises an upper prominence (31) originating from the upper cross wall or added onto it.

14. A device according to claim 8, whereby the means capable of exchanging heat comprises:
a lower prominence (6, 25) originating from the lower cross wall and a circular ring (12, 30) clamping said lower prominence,
and/or
an upper prominence (31) originating from the upper cross wall or added on to and a circular ring (33) clamping said upper prominence, these rings containing and/or being coated and/or being impregnated with biocompatible agents, medicated or non-medicated.

15. A device according to claim 8, whereby the heat source corresponds to an in situ heat generating system comprising a supersaturated and supercooled saline solution and a means (14) of triggering exothermal crystallisation of the salt(s) in the solution.

16. A device according to claim 8, whereby the heat source corresponds to an in situ heat generating system comprising a supersaturated and supercooled saline solution and a means (14) of triggering exothermal crystallisation of the salt(s) in the solution, said means (14) comprising a trigger diaphragm (15) which comprises a curved portion (16) in the general shape of an equilateral triangle with sides curved slightly inwards and with rounded vertices.

17. A device according to claim 8, whereby the heat source corresponds to an in situ heat generating system comprising a supersaturated and supercooled saline solution and a means (14) of triggering exothermal crystallisation of the salt(s) in the solution, said means (14) comprising a trigger diaphragm (15) the thickness of which varying from its centre towards its outside periphery as a function of the different tension zones thus created during when axial pressure is applied on this diaphragm to cause its deformation.

18. A device according to claim 8, whereby the heat source corresponds to an in situ heat generating system comprising a supercooled saline solution and a means (14) of triggering exothermal crystallisation of the salt(s) in the solution, said means (14) comprising a trigger diaphragm (15) which comprises a curved portion (16) itself comprising three slits (17) of equal length that extend symmetrically from its centre, each terminating in an office (18), these orifices being similar, and three similar grooves (20) located between the orifices and on a circumference with the same centre as said curved portion.
